(19) United States
(12) Patent Application Publication
(10) Pub. No.: US 2017/0023591 A1
(43) Pub. Date: Jan. 26, 2017

(54) Traumatic Brain Injury and Neurodegenerative Biomarkers, Methods, and Systems

(71) Applicant: Iron Horse Diagnostics, Inc., Scottsdale, AZ (US)

(72) Inventors: Robert Bowser, Scottsdale, AZ (US); Andreas Jeromin, Scottsdale, AZ (US); Gerry Shaw, Scottsdale, AZ (US)

(21) Appl. No.: 15/302,421

(22) PCT Filed: Apr. 7, 2015
(86) PCT No.: PCT/US15/24739

§ 371 Date: Oct. 6, 2016

Related U.S. Application Data
(60) Provisional application No. 61/976,380, filed on Apr. 7, 2014.

(51) Int. Cl. G01N 33/68

(52) U.S. Cl. CPC G01N 33/6896; G01N 2800/52; G01N 2800/2821; G01N 2800/2835; G01N 2800/285; G01N 2800/2814

(57) ABSTRACT

Biomarkers, methods, and systems for assessment of traumatic brain injury of different severities, as well as treatment efficacy and blood brain barrier or blood cerebrospinal fluid integrity and assessment of neurodegenerative conditions. The methods include detecting in a patient sample one or more of ubiquitin C-terminal hydrolase LI (UCH-L1), glial fibrillary acid protein (GFAP), aldehyde dehydrogenase 1 family member LI (ALDHILI), phosphorylated neurofilament heavy chain (pNFH), medium chain (NFM), or light chain (NFL), alpha-synuclein, visinin-like protein 1 (VILIP-1) and S100B.

### CROSS-REFERENCE TO RELATED APPLICATIONS

This application claims priority to U.S. Provisional Patent Application No. 61/976,380 filed on Apr. 7, 2014.

### TECHNICAL FIELD

This invention relates to biomarkers, methods, and systems for diagnosing and otherwise assessing traumatic brain injury and indications of neurodegeneration.

### BACKGROUND

Genomic advances over recent years have markedly improved our understanding of the molecular alterations that drive pathological outcomes. Technical innovations in proteomics have translated this knowledge into a rapid pace of discovery of potential biomarkers for the early detection and prognosis of different conditions.

Traumatic brain injury is a complex condition that results from mild to severe head injury in approximately 1.4 million US citizens that will suffer a brain injury this year, with between 1-4 million sports related concussions each year. Traumatic brain injury (TBI) and blast-related concussion are also a significant problem in the military, with over 300,000 diagnoses of TBI in the U.S. military since 2000. Early intervention and improved patient outcomes require objective assays to determine the severity of brain injury and prognosis.

Mild traumatic brain injury (mTBI) is currently defined as head trauma that results in one of the following: altered mental state for up to 24 hours (dazed, confused, disoriented), loss of consciousness for less than 30 minutes, or loss of memory for events immediately before or after the trauma. Initial assessment and diagnosis by clinicians is based on the Glasgow Coma Scale (GCS), which identifies TBI as mild, moderate or severe. While severe head trauma can be easily identified by a clinician, mTBI is a complex etiology that is often misdiagnosed and an objective biomarker-based standard for mild TBI, also known as concussion, does not exist.

Computerized axial tomography (CAT or CT) scans are often used in hospitals to identify brain injuries, though they are often not useful at detecting mTBI in which there is no obvious damage to the brain. Mild TBI with normal CT are distinguished from moderate TBI with bleeding in the TBI and abnormal CT scan. Imaging (CT or MRI) are subjective measures of brain injury and not sensitive or specific enough or too costly as clinical tools to identify degree of brain injury and are not easily available as screening tools.

Despite advancements, there remains a great need to identify biomarkers for mTBI in both the adult and pediatric population to utilize such biomarkers in methods and systems to improve rapid diagnosis of mTBI, assessment (e.g., response to treatment), and prognostic indications for patients with mTBI. There also is a need for protein markers of various neurodegenerative conditions.

### SUMMARY

This disclosure relates in one aspect to biomarkers for brain injury, in particular biomarkers correlated to the identification, assessment, and prognostic indications for traumatic brain injury.

In some embodiments, methods for determining brain injury in a subject (adult or pediatric) are provided. One method includes collecting a sample from the subject, measuring the presence or amount of one or more biomarkers indicative of traumatic brain injury in the sample, and comparing the levels of these biomarkers to predefined levels of the same biomarkers in patients with or without brain injury, wherein a correlation to one of the predefined levels provides a diagnosis.

In one preferred embodiment, the biomarkers are one or more of the following: ubiquitin C-terminal hydrolase L1 (UCH-L1), glial markers such as glial fibrillary acid protein (GFAP), aldehyde dehydrogenase 1 family member L1 (ALDH1L1) and other glial proteins, phosphorylated neurofilament heavy chain (pNFH), medium chain or light chain (NFM and light chain (NFL), alpha-synuclein, visinin-like protein 1 (VILIP-1) and S100B. The sample is typically blood or cerebrospinal fluid (CSF). The levels or concentrations of the biomarkers can be used to determine the onset of brain injury, diagnostic decisions and clinical management, monitor the progression of brain injury, or monitor the progression of a treatment for brain injury. These biomarkers will be useful for mTBI, in addition to more severe forms of TBI.

Other embodiments relate to detection or assessment of altered protein levels or abnormalities and to altered gene expression or splicing relating to frontotemporal lobar degeneration, vascular dementia, Pick's disease, neuromuscular disorders, and other neurodegenerative conditions.

Other features and advantages of the invention will be apparent from the following detailed description and figure, and from the claims.

### DESCRIPTION OF DRAWINGS

FIG. 1 depicts the biomarker UCH-L1 in blood differentiates TBI patients from healthy controls. Prospective single-center European study in the ED (67 TBIs, 60 controls (including trauma controls), standard of care (GCS, CT).

FIG. 2 depicts biomarker levels in blood samples from 15 mTBI patients. Many exhibit 2 of 3 biomarkers, suggesting a combination/panel of biomarkers would provide the optimal test results.

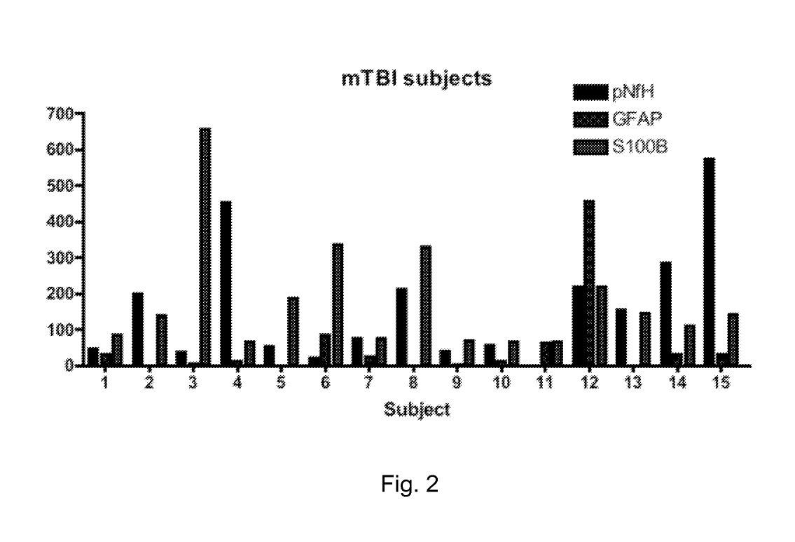

FIG. 3 depicts serum and CSF levels of UCH-L1 over 7 days for severe TBI patients in comparison to controls. Data shown are the Mean +/−SEM concentrations. For controls, only a single time point is shown as a bar on the far left.

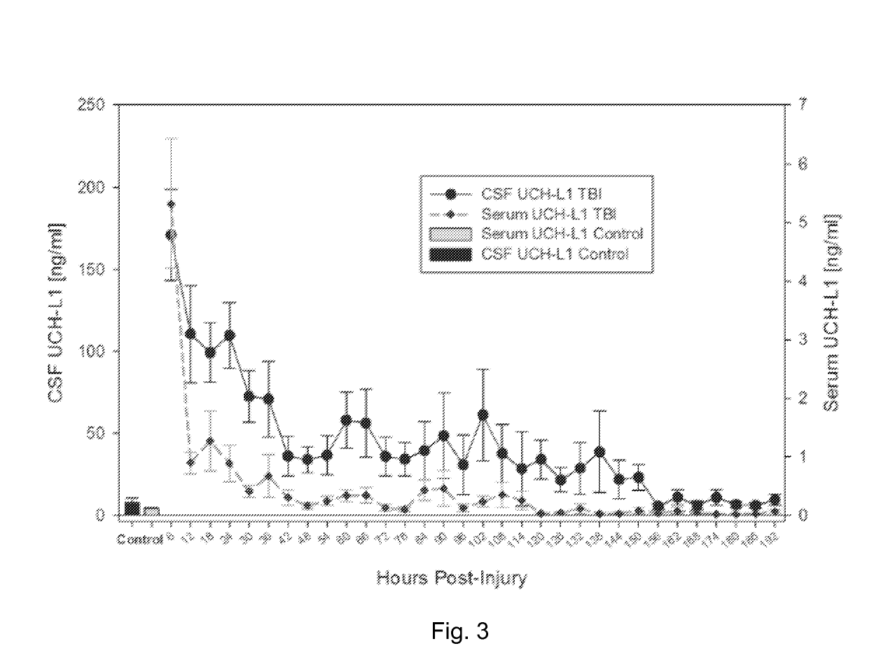

FIG. 4 depicts the biomarkers UCH-L1 and phosphorylated neurofilament heavy chain in blood clinically separate CT normal from abnormal subjects in the overall clinical cohort and also within 4 hrs post-injury, supporting their use as diagnostic biomarkers in TBI. The blood levels of UCHL1 and pNFH in 67 TBI patients (Top). Both biomarkers differentiate CT normal (mTBI) from CT abnormal patients (moderate TBI) (Bottom). This distinction can be made within 4 hours post injury.

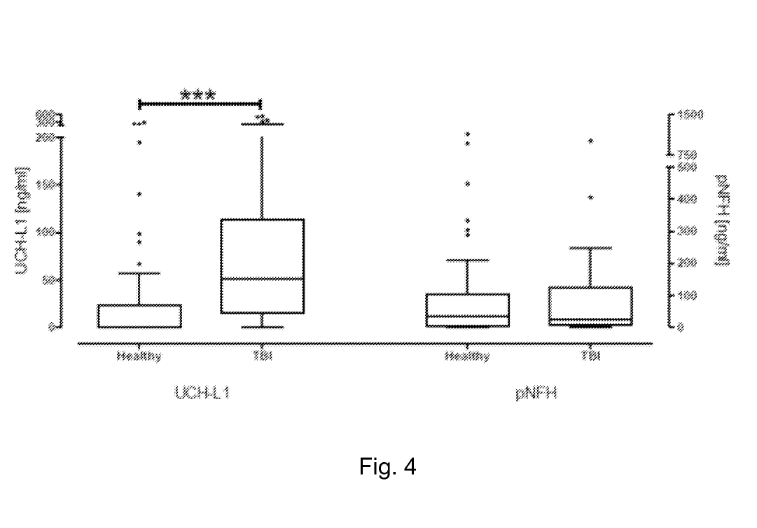

FIG. 5 depicts UCH-L1 and pNFH can differentiate subtypes of TBI based on CT, while phosphorylated neurofilament heavy chain levels in blood differentiate subjects with normal CT, subarachnoid hemorrhage (SAH), and subdural hematoma (SDH).

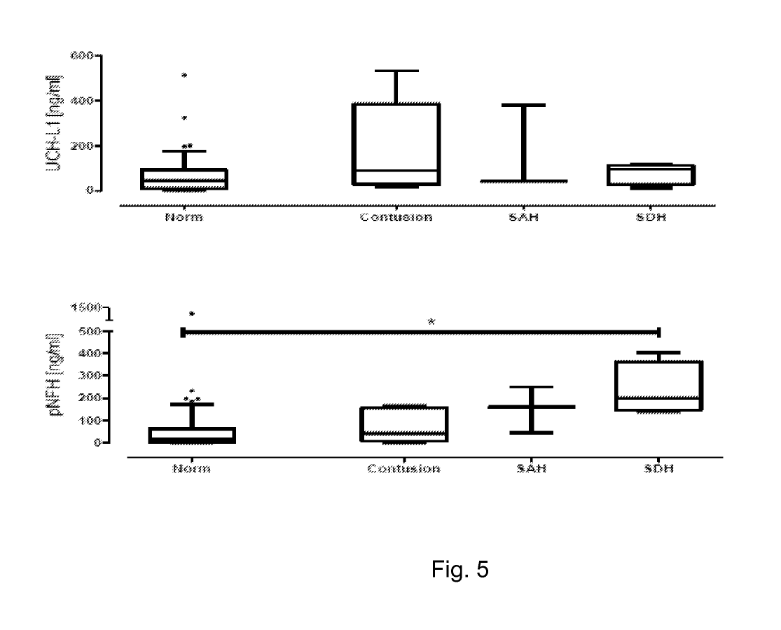

### DETAILED DESCRIPTION

During a brain injury, damage to the brain occurs due to rapid acceleration and deceleration, an overpressure wave associated with a blast, or penetration with a foreign object. The blood-brain barrier and vasculature may be damaged or disrupted due to these injuries, resulting in direct access of blood to the brain tissue and exchange of protein components within the brain and the circulating blood. Proteins released into the blood from the central nervous system may represent biomarkers for brain injury as well as various neurodegenerative conditions.

The term “sample,” as used herein refers to biological material isolated from a human and or animal. The sample can contain any suitable biological material a particular tissue or biological fluid. The sample can be isolated from any suitable tissue or biological fluid. In this respect, the sample can be blood, blood serum, plasma, urine, CSF or spinal cord tissue. In that TBI affects the central nervous system, the sample preferably is isolated from tissue or biological fluid of the central nervous system (CNS) (i.e., brain and spinal cord). In a preferred embodiment, the sample is isolated from the blood.

The sample can be obtained in any suitable manner known in the art, such as, for example, by biopsy, blood sampling, urine sampling, lumbar puncture (i.e., spinal tap), ventricular puncture, and cisternal puncture. In a preferred embodiment, the sample is obtained by lumbar puncture, which also is referred to as a spinal tap or CSF collection. Lumbar puncture involves insertion of a spinal needle, usually between the 3rd and 4th lumbar vertebrae, into the subarachnoid space where CSF is collected. In instances where there is lumbar deformity or infection which would make lumbar puncture impossible or unreliable, the sample can be collected by ventricular puncture or cisternal puncture. Ventricular puncture typically is performed in human subjects with possible impending brain herniation. Ventricular puncture involves drilling a hole in the skull and inserting a needle directly into the lateral ventricle of the brain to collect CSF. Cisternal puncture involves insertion of a needle below the occipital bone (back of the skull), and can be hazardous due to the proximity of the needle to the brain stem.

Many neurodegenerative diseases, such as Alzheimer's disease, Parkinson's disease, Huntington's disease, and ALS are characterized by the accumulation or presence of protein abnormalities which contribute to the disease phenotype. In addition to proteins, metabolite abnormalities in the sample can be used as an indicator of a diseased state. Thus, embodiments herein related to detection or assessment of altered protein levels or abnormalities and to altered gene expression or splicing, including but not limited to frontotemporal lobar degeneration, vascular dementia, Pick's disease, neuromuscular disorders.

The terms “individual,” “host”, “subject”, and “patient” are used interchangeably herein, and refer to a mammal, including, but not limited to, humans, rodents such as mice and rats, and other laboratory animals.

The term “biomarker” refers to an organic molecule produced by an organism that is indicative or correlative of a disease state or condition. Biomarkers include, but are not limited to protein, metabolites, post-translationally modified proteins, etc.

We have generated antibodies and developed a specific and high-sensitivity assay for UCH-L1. The ELISA has a detection range from 10 ng/mL down to 20 pg/mL. The assays use EnCor™ monoclonal antibody MCA-BH7 for capture and rabbit polyclonal RPCA-UCHL1 for detection.

The prototype ELISA type assay for VSNL1/Vilip1 has a range of detection from 100 ng/mL to 1 ng/mL, again with sufficient sensitivity to detect the elevated levels of this protein expected to occur in CSF, plasma, and serum based on published data. This uses the monoclonal MCA-3A9 for capture and rabbit polyclonal RPCA-VSNL1 for detection. We previously published the extensive characterization of antibodies and an ELISA for SNCA/α-synuclein which could reliably detect this protein in the range from 100 ng/mL to ˜1 ng/mL. This uses the monoclonal MCA-2A7 for capture and the rabbit polyclonal RPCA-aSyn for detection.

The embodiments will be further described in the following examples, which do not limit the scope of the invention defined by the claims.

### EXAMPLES

Methods for diagnosing mTBI. One embodiment includes measuring biomarker levels in a sample obtained from a subject and correlating levels of these biomarkers to predefined levels of biomarkers in patients known to have mTBI, moderate or severe TBI, or no brain injury. The biomarkers include one or more of the following: ubiquitin C-terminal hydrolase L1 (UCH-L1), glial markers such as glial fibrillary acid protein (GFAP), aldehyde dehydrogenase 1 family member L1 (ALDH1L1) and other glial proteins, phosphorylated neurofilament heavy chain (pNFH), medium chain or light chain (pNFM and light chain (pNFL), alpha-synuclein, visinin-like protein 1 (VILIP-1) and S100B.

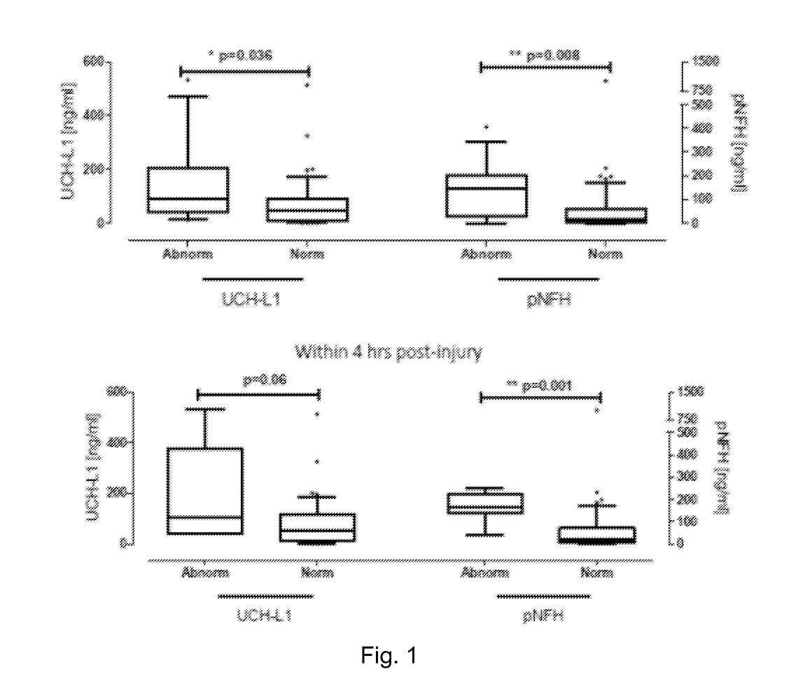

FIG. 1 depicts different levels of UCH-L1 and pNFH in serum in a cohort of 67 subjects, who presented themselves in the Emergency department for clinical assessment and CT scan. Blood samples were taken upon arrival at the Emergency department and proteins measured by immunoassay. A normal CT scan is typical for mTBI and an abnormal CT scan typical for a moderate or severe TBI. The Top panel shows UCH-L1 and pNFH protein levels, demonstrating that both UCH-L1 and pNFH exhibit increased levels in patients with abnormal CT scan versus normal CT scan.

Those with a normal CT scan still exhibit higher UCH-L1 or pNFH levels in the blood versus healthy controls. The lower panel depicts a comparison of UCH-L1and pNFH levels in serum in subjects that arrived to the emergency department within 4 hrs post-injury. Both UCH-L1 and pNFH levels were significantly different in the mild (CT normal) vs. moderate (CT abnormal) TBI group levels. Defining cut-off values for these biomarkers either alone or in combination generates a diagnosis of mTBI and discriminates between mild and moderate TBI, which will enable differential clinical management.

The methods for measuring the concentrations of biomarkers for the embodiments herein described include immunoassays or systems utilizing mass spectrometry. Specific immunoassays (ELISAs) for the different biomarkers have been developed, using the Meso-Scale Discovery platform and known immunoassay conditions. These are based on the use a biomarker-specific mouse monoclonal capture antibody and the use of polyclonal detection antibody conjugated to ruthenium red for detection by electro-chemiluminescence. Calibrators for the immunoassays were purified from bovine spinal cord (for pNFH) and/or expressed as recombinant protein (for UCH-L1).

For blood detection of neurofilament proteins, treating the blood sample with Urea reduces protein aggregation, which enhances the immunoassay measurement in some samples and therefore improves overall results.

The mass spectrometry methods would include tryptic digestion (or digestion with another well known enzyme) and then liquid chromatography tandem mass spectrometry to identify and sequence the peptides to identify each of the biomarkers. Quantitative mass spectrometry can be used to accurately quantify each peptide within the biofluid.

Methods to monitor therapeutic treatment of a TBI. One embodiment includes measuring biomarker levels in samples obtained over time from a TBI subject both before and after therapy and correlating changes in the levels of these biomarkers over time. The biomarkers include one or more of the following: ubiquitin C-terminal hydrolase L1 (UCH-L1), glial markers such as glial fibrillary acid protein (GFAP), aldehyde dehydrogenase 1 family member L1 (ALDH1L1) and other glial proteins, phosphorylated neurofilament heavy chain (pNFH), medium chain or light chain (pNFM and light chain (pNFL), alpha-synuclein, visinin-like protein 1 (VILIP-1) and S100B.

Methods to measure blood-brain barrier integrity or blood-cerebrospinal fluid integrity. A preferred method includes measuring biomarker levels in samples from subject from blood or CSF. The biomarkers include one or more of the following: ubiquitin C-terminal hydrolase L1 (UCH-L1), glial markers such as glial fibrillary acid protein (GFAP), aldehyde dehydrogenase 1 family member L1 (ALDH1L1) and other glial proteins, phosphorylated neurofilament heavy chain (pNFH), medium chain or light chain (pNFM and light chain (pNFL), alpha-synuclein, visinin-like protein 1 (VILIP-1) and S100B.

Methods to measure injury mechanisms, region of injury, heterogeneity and outcomes due to brain injury. Our preferred method includes measuring biomarker levels in samples from subject from blood or CSF. The biomarkers include one or more of the following: ubiquitin C-terminal hydrolase L1 (UCH-L1), glial markers such as glial fibrillary acid protein (GFAP), aldehyde dehydrogenase 1 family member L1 (ALDH1L1) and other glial proteins, phosphorylated neurofilament heavy chain (pNFH), medium chain or light chain (pNFM and light chain (pNFL), alpha-synuclein, visinin-like protein 1 (VILIP-1) and S100B.

TBI heterogeneity arises from different proteins present or absent in the blood of TBI patients and can be used to differentiate patients and assist in clinical management. Prognostic indicators of TBI are identified by correlating biomarker levels to predefined levels of biomarkers in patients with known clinical outcomes, using biomarker cut-off values for prognostic indications.

It should be noted that the embodiments herein may further include monitoring temporal kinetics and processing of one or more biomarkers as a measure of treatment efficacy and outcome.

Thus, it is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessment of traumatic brain injury of unknown severity, comprising detecting in a patient sample one or more biomarker(s) selected from the group consisting of ubiquitin C-terminal hydrolase L1 (UCH-L1), glial fibrillary acid protein (GFAP), aldehyde dehydrogenase 1 family member L1 (ALDH1L1), phosphorylated neurofilament heavy chain (pNFH), medium chain (NFM), or light chain (NFL), alpha-synuclein, visinin-like protein 1 (VILIP-1) and S100B, and comparing a detection result from said sample to a control for known traumatic brain injury severity for said one or more of said biomarker(s).

2. The method of claim 1, wherein said detecting comprises an antibody-capture method.

3. The method of claim 1, further including detecting a breakdown product or other processed variant of one or more of said ubiquitin C-terminal hydrolase L1 (UCH-L1), glial fibrillary acid protein (GFAP), aldehyde dehydrogenase 1 family member L1 (ALDH1L1), phosphorylated neurofilament heavy chain (pNFH), medium chain (NFM), or light chain (NFL), alpha-synuclein, visinin-like protein 1 (VILIP-1) and S100B.

4. The method of claim 3, wherein a detection result from said detecting a breakdown product or other processed variant is followed by comparison to a control for a known neurodegeration disorder or disease.

5. The method of claim 4, where said neurodegenerative condition is selected from the group consisting of Alzheimer's disease, Parkinson's disease, Huntington's disease, ALS, frontotemporal lobar degeneration, vascular dementia, and Pick's disease.

6. The method of claim 1, wherein said biomarker(s) comprise ubiquitin C-terminal hydrolase L1 (UCH-L1) and phosphorylated neurofilament heavy chain (pNFH).

7. The method of claim 1, wherein said detecting comprises processing said sample with mass spectrometry.

8. A method of detecting a neurodegenerative condition or disease, comprising detecting one or more protein abnormalities in one or more biomarkers a patient sample, said one or more biomarkers selected from the group consisting of ubiquitin C-terminal hydrolase L1 (UCH-L1), glial fibrillary acid protein (GFAP), aldehyde dehydrogenase 1 family member L1 (ALDH1L1), phosphorylated neurofilament heavy chain (pNFH), medium chain (NFM), or light chain (NFL), alpha-synuclein, visinin-like protein 1 (VILIP-1) and S100B, wherein a detection result from said detecting a protein abnormality is followed by comparison to a control for a known neurodegeration disorder or disease.

9. The method of claim 8, further comprising measuring temporal kinetics and processing of said biomarkers as measure of one or more of injury mechanism, region of injury, brain injury heterogeneity, and outcome.

10. The method of claim 8, wherein said detecting comprises an antibody-capture method.

11. The method of claim 8, wherein said detecting one or more protein abnormalities comprises detecting a breakdown product or other processed variant of said one or more biomarkers selected from the group consisting of ubiquitin C-terminal hydrolase L1 (UCH-L1), glial fibrillary acid protein (GFAP), aldehyde dehydrogenase 1 family member L1 (ALDH1L1), phosphorylated neurofilament heavy chain (pNFH), medium chain (NFM), or light chain (NFL), alpha-synuclein, visinin-like protein 1 (VILIP-1) and S100B.

12. The method of claim 8, wherein said detecting comprises processing said sample with mass spectrometry.

13. The method of claim 8, where said neurodegenerative condition or disease is selected from the group consisting of Alzheimer's disease, Parkinson's disease, Huntington's disease, ALS, frontotemporal lobar degeneration, vascular dementia, and Pick's disease.

14. A method of assessing therapeutic treatment of a traumatic brain injury, comprising detecting in a sample from a patient undergoing treatment for said injury one or more biomarkers selected from the group consisting of ubiquitin C-terminal hydrolase L1 (UCH-L1), glial fibrillary acid protein (GFAP), aldehyde dehydrogenase 1 family member L1 (ALDH1L1), phosphorylated neurofilament heavy chain (pNFH), medium chain (NFM), or light chain (NFL), alpha-synuclein, visinin-like protein 1 (VILIP-1) and S100B, wherein a detection result is followed by comparison to a prior assessment of one or more said biomarkers in a sample from said patient.

15. The method of claim 14, further comprising monitoring temporal kinetics and processing of said biomarkers as a measure of treatment efficacy and outcome.

16. The method of claim 14, wherein said detecting comprises an antibody-capture method.

17. The method of claim 14, wherein said detecting comprises processing said sample with mass spectrometry.
